(19) United States
(12) Patent Application Publication
(10) Pub. No.: US 2023/0371921 A1
(43) Pub. Date: Nov. 23, 2023

(54) METHOD FOR ANALYZING ULTRASOUND DATA OBTAINED BY PASSING THROUGH MULTIPLE LAYERS

(71) Applicant: NATIONAL CANCER CENTER, Goyang-si (KR)

(72) Inventors: Dae Woo PARK, Goyang-si (KR); Dong Chan PARK, Goyang-si (KR)

(73) Assignee: NATIONAL CANCER CENTER, Goyang-si (KR)

(21) Appl. No.: 18/044,896

(22) PCT Filed: Oct. 19, 2021
(86) PCT No.: PCT/KR2021/014557

§ 371 Date: Mar. 10, 2023

(30) Foreign Application Priority Data
Oct. 21, 2020 (KR) 10-2020-0136576

(51) Int. Cl. A61B 8/08; A61B 8/00

(52) U.S. Cl. CPC A61B 8/0858; A61B 8/5269; A61B 8/58

(57) ABSTRACT

The present invention relates to a method for analyzing ultrasound data obtained by passing through multiple layers, comprising the steps of: reducing noise in the ultrasound data; identifying an interface between layers by identifying the peak of the ultrasound data after removal of the noise; and differentiating each layer by identifying an attenuation coefficient of the ultrasound data after removal of the noise.

### TECHNICAL FIELD

The present disclosure relates to a method for analyzing ultrasound data obtained by passing through multiple layers.

### BACKGROUND ART

In examining health conditions, it is essential to figure out the body's composition accurately. Changes in body composition (organization) are associated with many diseases, and the occurrence of a disease or health condition may be identified by examining the changes in body composition.

Ultrasound methods are used for measuring the thickness of muscle, fat, and bone among body compositions. Ultrasound has an advantage in terms of easy inspection and convenience of use.

However, existing ultrasound methods have a problem in that they are not capable of clearly distinguishing layers due to other reflection data such as those from intramuscular fat.

### DISCLOSURE

### Technical Problem

Therefore, an object of the present disclosure is to provide a method for analyzing ultrasonic data obtained by passing ultrasound through multiple layers.

### Technical Solution

To achieve the object above, a method for analyzing ultrasound data by passing ultrasound through multiple layers according to the present disclosure comprises reducing noise in the ultrasound data; identifying an interface between layers by detecting the peak of the ultrasound data after removal of the noise; and identifying (classifying) each layer by estimating an attenuation coefficient of the ultrasound data after removal of the noise.

The reducing the noise may include removing in-band noise; removing out-of-band noise; preserving a peak position in the band-pass filtered ultrasound data; and converting the ultrasound data into envelope data.

The preserving the peak position may be performed by a zero-phase filtering method, and the converting into envelope data may be performed by Hilbert transform.

The identifying the interface between layers includes determining the local maximum value, and the local maximum value may be obtained using minimum peak distance (MPD) and minimum peak prominence (MPP).

After the identifying the interface between the layers, the method may further include determining the thickness of each layer using ultrasonic speed between the interfaces.

The multiple layers may include at least two layers from among bone, muscle, and fat.

The identifying each layer may further include correcting the diffraction characteristics of a probe using ultrasound data obtained from a phantom whose attenuation coefficient is known.

### Advantageous Effects

According to the present disclosure, a method for analyzing ultrasound data obtained by passing ultrasound through multiple layers is provided.

### DESCRIPTION OF DRAWINGS

FIG. 1 is a flow diagram illustrating a method for analyzing ultrasound data according to one embodiment of the present disclosure.

FIGS. 2a to 2c illustrate a sample preparation process in an experiment according to the present disclosure.

FIG. 3 illustrates a process for analyzing ultrasound data in an experiment according to the present disclosure.

FIGS. 4a to 4e illustrate step-by-step analysis of ultrasound data and an MR image in an experiment according to the present disclosure.

FIGS. 5a and 5b are graphs illustrating a correlation between thickness obtained by analysis of ultrasound data and thickness obtained from an MR image in an experiment according to the present disclosure.

### MODE FOR DISCLOSURE

In what follows, the present disclosure will be described in detail with reference to appended drawings.

The appended drawings are only an example introduced to describe the technical principles of the present disclosure in more detail; thus, the technical principles of the present disclosure are not limited to the appended drawings. In the appended drawings, thickness or length of each part may have been exaggerated for the convenience of descriptions.

The embodiments described above are introduced to illustrate the present disclosure, and the present disclosure is not limited to the specific embodiments. It should be understood by those skilled in the art to which the present disclosure belongs that the present disclosure may be modified in various ways from the embodiments; therefore, the technical scope of the present disclosure should be determined by the appended claims.

In the following description, a method for identifying animal or human tissue, in particular, identifying muscle/fat/bone and determining the thickness thereof, by analyzing ultrasound data is illustrated; however, the present disclosure is not limited to the specific illustration. The analysis method of the present disclosure may be used to analyze ultrasound data obtained by passing ultrasound through various types of multiple layers.

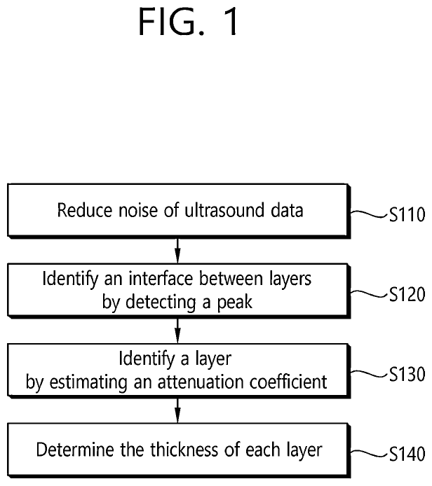

A method for analyzing ultrasound data according to the present disclosure will be described with reference to FIG. 1. FIG. 1 is a flow diagram illustrating a method for analyzing ultrasound data according to one embodiment of the present disclosure.

Ultrasound data to which the present disclosure may be obtained by bringing an ultrasonic probe (transducer) into contact with a body part such as an arm or a leg.

First, noise in the ultrasound data is reduced S110.

Noise reduction includes, but is not limited to, (1) in-band noise removal, (2) out-of-band noise removal, (3) preserving peak positions in band-pass filtered ultrasound data, and (4) converting ultrasound data into envelope data. The order of the steps may be changed.

Each of the steps may be performed using a method known to the public; for example, peak position preservation may be performed by a zero-phase filtering method, and envelope data conversion may be performed by Hilbert transform.

Next, after removing the noise, the peak of the ultrasound data is identified to determine the interface between the layers S120.

The step above includes the step of finding a local maximum. The local maximum value may be obtained using, but is not limited to, minimum peak distance (MPD) and minimum peak prominence (MPP).

Next, after noise removal, each layer is identified by estimating the attenuation coefficient of the ultrasound data S130.

The attenuation coefficient may be obtained by, but is not limited to, the following methods.

First, ultrasound data in the time domain is transformed into the frequency domain. Then, the centroid is calculated at each tissue depth in the frequency domain. Next, a change of the centroid according to tissue depth, namely, a frequency shift is calculated. Finally, the attenuation coefficient is obtained by applying linear regression to the change of the centroid according to the depth within the region of interest (ROI).

The attenuation coefficient has a unique range for each layer, and each layer is identified by comparing the unique attenuation coefficient with an identified attenuation coefficient.

In this step, the diffraction characteristics of a probe may be corrected using the ultrasonic data obtained from a phantom whose attenuation coefficient is known.

Finally, the thickness of each layer is determined S140. The thickness of each layer may be obtained using ultrasonic speed between the identified interfaces.

In the descriptions above, the order of steps after noise removal according to the present disclosure may be changed. For example, the interfaces may be identified after estimating the attenuation coefficient, or the attenuation coefficient may be obtained after determining the thickness.

According to the present disclosure, each layer may be identified, and the thickness thereof may be determined; also, the thickness of each interface may be determined by excluding other reflection data, such as those from intramuscular fat, through the identification of a true peak and each layer using the local maximum of ultrasound data. Also, fat and muscle mass measurements may be performed simultaneously from ultrasound data.

In what follows, the present disclosure will be described in detail through an experiment.

Preparation of Measurement Target

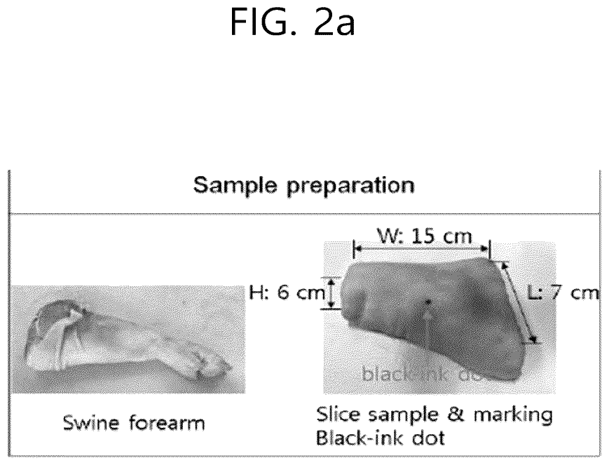

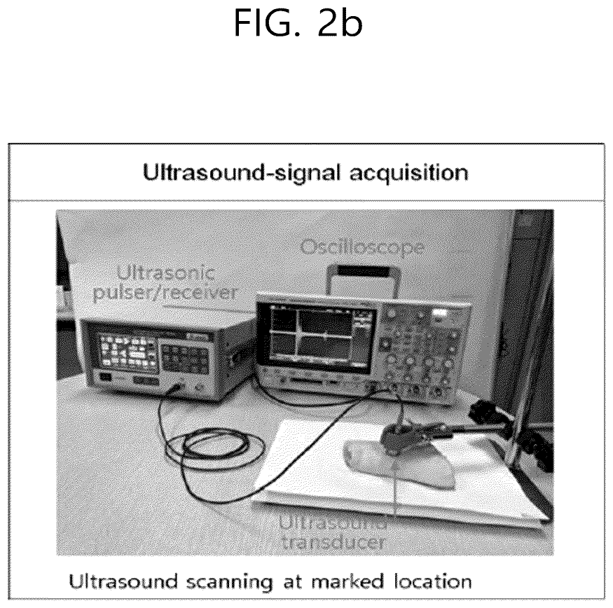

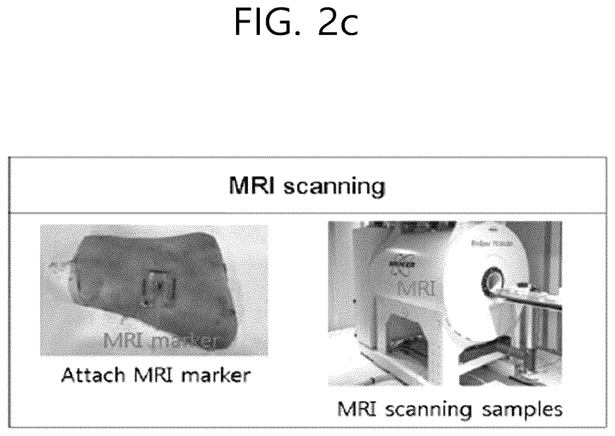

FIGS. 2a, 2b, and 2c show the preparation of the measurement target in sequence. Three forelegs of pigs were purchased from a butcher shop, and 16 samples of the forelegs were obtained through cutting. Each sample had a predetermined size and was prepared to contain subcutaneous fat, muscle, and bone. Ultrasonic measurement points were marked on each sample with black ink.

Acquisition of Ultrasound Data

For 16 samples, ultrasound data were obtained at the locations marked with black ink.

A 2.25-MHz single-element focused transducer (BioSono Inc., USA) was excited with an ultrasonic pulser/receiver (Model XTR-2020, MKC Inc., Korea), the obtained ultrasonic data was digitized at 20-MS/s sampling rate using an oscilloscope (Model DSO1012A, Keysight Technologies, Korea), and the digitized ultrasonic data was stored in a computer. Each location was scanned five times, and the data was stored separately.

Analysis of Ultrasonic Data

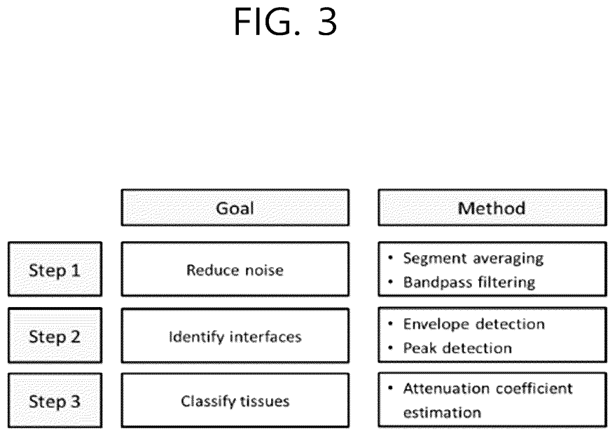

Analysis of ultrasonic data was conducted using MATLAB (Mathworks, Natick, USA), and detailed descriptions of the analysis will be provided with reference to FIG. 3.

FIG. 3 is a conceptual diagram of data processing performed in the present experiment.

Step 1

Segment averaging is performed to reduce in-band noise for M segments.

$$s ( n ) = \frac{1}{M} {\sum }_{m = 0}^{M - 1} {s}_{m} ( n ) , n = 0 , 1 , … , N - 1$$

In the equation above, s(n) is an average of ultrasound data (signal), sₘ(n) represents m-th ultrasound segment, M represents the number of pulses for each measurement, and N represents the number of digitized pulses during a pulse repetition interval. After segment averaging, out-of-band noise was suppressed using a 5th-order Butterworth bandpass filter with cutoff frequencies of 500 kHz and 5 MHz, and zero-phase filtering was used to preserve peak positions in the ultrasound data. Finally, Hilbert transform and log compression were performed to obtain log-compressed envelope signals.

Step 2

Tissue interfaces are identified by determining the local maximum within the log-compressed envelope signal. The local maximum algorithm requires the minimum peak distance (MPD) and the minimum peak prominence (MPP). The MPD determines the minimum separation between detected peaks; therefore, the MPP has to be greater than the peak duration. The prominence of a peak represents the peak's intrinsic height distinguished from other peaks. Using the MPD of 1 μm and the MPP of 10 dB, a strong peak generated by reflection from the tissue boundary and a weak peak generated by envelope fluctuation due to backscattering may be distinguished. Using the detected strong peak, the ultrasonic wave travel time between the interfaces among subcutaneous fat, muscle, and bone was determined. Then, subcutaneous fat and muscle thicknesses were obtained by multiplying the speed of sound (1547 m/s) passing through the tissue by half the ultrasonic wave travel time.

Step 3

The subcutaneous fat, muscle, and bone were identified by estimating the local attenuation coefficients of the respective tissues. The attenuation coefficient was calculated using a frequency-shift estimator with a window size of 1 μs and five independent ultrasound lines. The diffraction effect of the transducer was corrected using a uniform reference phantom having an attenuation coefficient of 0.511 dB/cm/MHz measured from a tissue-like phantom (PeripheralVascular Doppler FlowPhantom; Model 524; ATS Laboratories, USA).

Verification Using MRI

MRI data were obtained for each sample at the same locations as the ultrasound measurements using Bruker Biospec 7T system (BioSpec 70/20 USR; Bruker, Germany).

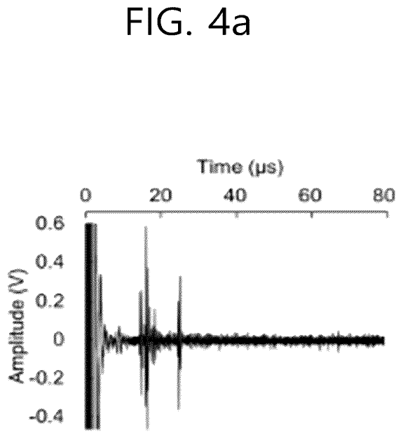

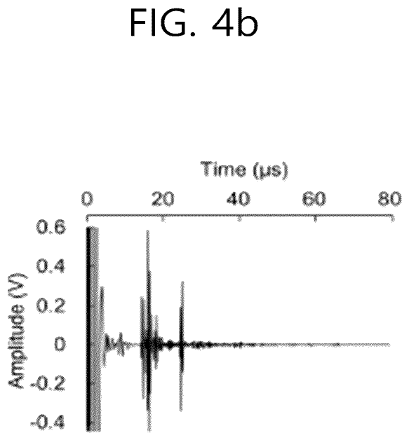

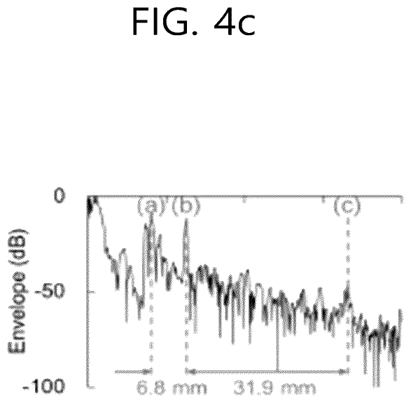

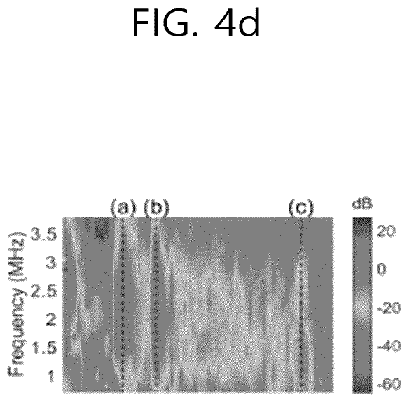

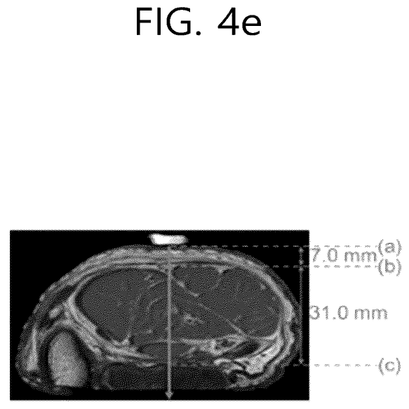

FIGS. 4a to 4e show a process of processing ultrasound data and the corresponding MR image. FIG. 4a shows ultrasound data as ultrasound wave travel time against voltage. FIG. 4b shows ultrasound data after segment averaging and band-pass filtering. FIG. 4c shows log-compressed envelope data, including three prominence peaks at (a) 16.2 s, (b) 25.0 s, and (c) 66.2 s. FIG. 4d shows a frequency map against time including three peaks. As a result of the calculation, subcutaneous fat and muscle thicknesses were 6.8 mm and 31.9 mm, respectively. FIG. 4e shows an MRI scan of the corresponding sample. The yellow line is the direction of the ultrasound measurement. It may be confirmed that the thicknesses of subcutaneous fat and muscle obtained by MRI are similar to those obtained by processing the ultrasound data.

The attenuation coefficients of subcutaneous fat, muscle, and bone for 16 samples are shown in Table 1 below.

TABLE 1

| Tissue | attenuation coefficient(dB/cm/MHz) |
|---|---|
| fat | 1.8859 ± 0.3599 |
| muscle | 1.0982 ± 0.3405 |
| bone | 4.5703 ± 0.8298 |

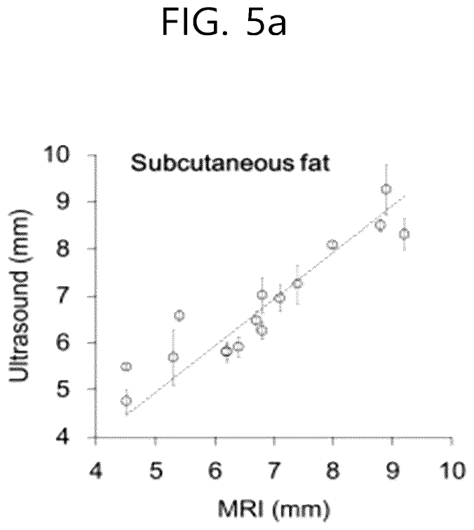

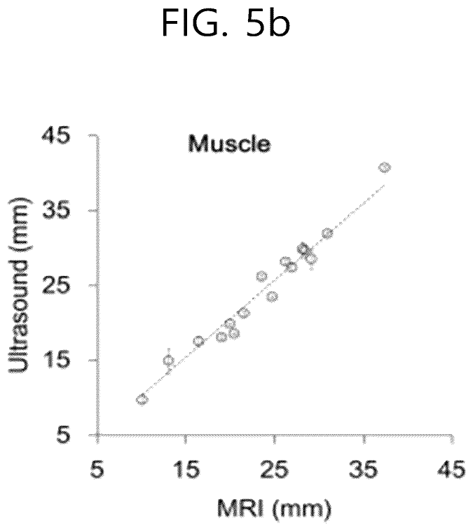

FIGS. 5a and 5b show comparisons between tissue thickness obtained from MRI and the thickness determined through analysis of ultrasound data. It may be checked that the correlation between the value obtained from MRI and the value obtained through ultrasound data analysis is very high.

1. A method for analyzing ultrasound data by passing ultrasound through multiple layers comprising:
reducing noise in the ultrasound data;
identifying an interface between layers by detecting the peak of the ultrasound data after removal of the noise; and
identifying each layer by estimating an attenuation coefficient of the ultrasound data after removal of the noise.

2. The method of claim 1, wherein the reducing the noise includes
removing in-band noise;
removing out-of-band noise;
preserving a peak position in the band-pass filtered ultrasound data; and
converting the ultrasound data into envelope data.

3. The method of claim 2, wherein the preserving the peak position is performed by a zero-phase filtering method, and the converting into envelope data is performed by Hilbert transform.

4. The method of claim 1, wherein the identifying the interface between layers includes determining the local maximum value, wherein the local maximum value is obtained using minimum peak distance (MPD) and minimum peak prominence (MPP).

5. The method of claim 1, further including:
determining the thickness of each layer using ultrasonic speed between the interfaces after the identifying the interface between the layers.

6. The method of claim 1, wherein the multiple layers include at least two layers from among bone, muscle, and fat.

7. The method of claim 1, wherein the identifying each layer further includes correcting diffraction characteristics of a probe using ultrasound data obtained from a phantom whose attenuation coefficient is known.
